(19) United States
(12) Patent Application Publication
(10) Pub. No.: US 2006/0127427 A1
(43) Pub. Date: Jun. 15, 2006

(54) Surface coated abrasive material for cosmetic products

(76) Inventors: Joseph J. Vernice, Shirley, NY (US); Kimberly J. Sweeney, Melville, NY (US)

Correspondence Address: Evelyn M. Sommer;Room 825, 250 Park Avenue, New York, NY, 10022, US

(21) Appl. No.: 11/011,974

(22) Filed: Dec. 15, 2004

(51) Int. Cl. A61K 8/19

(52) U.S. Cl. 424/401

(57) ABSTRACT

Composite particle comprising a water-soluble abrasive material or core and a shell comprising a coating which is substantially insoluble in aqueous media during storage and prior to use but which becomes substantially water soluble upon activation prior to or during use. Activation may include application of substantially large quantities of water in use or adjustment of the pH of the aqueous median just prior to or during use. Compositions containing the composite materials and methods for using them to exfoliate skin are displaced.

### FIELD OF THE INVENTION

The present invention generally relates to a surface coated abrasive material for cosmetic products. In particular, the invention relates to a water-soluble abrasive material coated with a coating material which is not water-soluble in the cosmetic composition, but which is rendered water-soluble during and after use. More particularly, the invention relates to a cosmetic composition containing the above-mentioned surface coated abrasive material for exfoliating the skin.

### BACKGROUND OF THE INVENTION

The skin of the human body periodically requires deep cleansing in order to remove there from sebaceous secretions, soil deposits and dead skin resulting from desquamation of the epidermal tissue. Exfoliation can be accomplished by the use of a rough cleaning rag, a natural sponge or an exfoliant composition.

While not wishing to be bound by the following skin treatment theories, it is generally believed that exfoliation improves skin cleansing by helping to mechanically remove dirt and oil from the skin. Exfoliation also is believed to aid the process of desquamation. Desquamation is a natural process by which comeocytes are removed from the stratum corneum, which is the top layer of skin cells. Exfoliation aids in removing the flaky comeocytes that are ready to detach from the stratum corneum, and so promotes smoother, less flaky skin. Other potential health benefits to exfoliation in addition to improved scale (flake) removal and oil removal, as suggested above, are reduction in bacteria on the skin, and increased blood flow to the skin due to the mechanical stimulation. While exfoliation is generally carried out for cosmetic purposes such as cleansing the skin and facilitating the penetration of cosmetics, its use for paint removal and in methods of medical treatment, such as treatment of acne (U.S. Pat. No. 3,092,111) and facilitation of the penetration of dermopharmaceutical products has also been proposed.

Numerous exfoliating abrasives have been known in the art, including hydrogenated fats, inorganic salts such as sodium citrate and tricalcium orthophosphate, synthetic polymers such as polyethylene powders and granulated particles or organopolysiloxane, vegetable matter such as the endocarp of apricot, peach and walnut seeds, almond flesh and wood flour, animal matter such as powdered crab shell and finely ground minerals such as silica, glacial limestone, aluminum oxide, or talc. Examples and references for the use of these exfoliating abrasives can be found in U.S. Pat. Nos. 4,284,533; 4,352,678; 4,704,222; 4.911,857; 4.957,747; 4,992,476; 5,679,326; 5,824,324; 5,891,449; 6,294,179; 6,290,976; 6,664,217.

The water insoluble abrasive particles such as minerals and walnut shells have been used widely in water-based cosmetic products and they are generally expected to be removed from the skin by wiping or rinsing with water after application to the parts of the body to be cleaned. While compositions containing these water-insoluble particles are generally effective in exfoliating the skin, some of the particles can remain in the pores of the skin and their removal from the skin is slow and often never complete. Further the water-insoluble particles can leave a gritty residue in the bath tub, shower or sink basin and the particles carried by water into the drain could contribute to blockage in the sewage system over a long period of time.

To overcome the difficulty of removal and disposal of the water-insoluble particulate abrasives, it is known in the art to use water-soluble abrasive materials dispersed in an oily phase, such as that taught by U.S. Pat. No. 4,673,526. Although the abrasive materials can be easily removed and disposed of by washing with water, the requirement of an oil-based product is sometimes difficult, uneconomical or undesirable to meet.

U.S. Pat. No. 4,048,123 teaches the use of sparingly soluble salts in an aqueous composition. The salt is expected to dissolve at a moderate rate rather than substantially instantaneously upon contact with a large amount of water during use. The removal of the salt from skin and the disposal of the salt in the sewage system are taken care of by prolonged water flow. This strategy, while it works, significantly limits the choices of abrasive materials that can be used and therefore may not be able to meet the requirements for a certain application, such as the economy of material.

There is therefore a practical need to for a method for using substantially any available water-soluble abrasive materials in aqueous compositions for purposes such as skin exfoliation.

It is therefore an object of this invention to provide an abrasive material which is only soluble in water upon activation.

It is another object of this invention to provide an abrasive material which has a water-soluble abrasive core and a shell or coating which is insoluble in aqueous media during storage and prior to use, and becomes water-soluble upon activation.

It is a further object of this invention to provide an abrasive material which is only soluble in water upon a change in the properties of the media, such as pH value of the media.

It is yet another object of this invention to provide an abrasive material with controlled water solubility for use in a water-based cosmetic composition for the purpose of exfoliating skin.

### SUMMARY OF THE INVENTION

In accordance with the invention composite particles comprising a water-soluble abrasive material as core, and a shell comprising a coating which is substantially insoluble in the aqueous media during storage and prior to use but which becomes substantially water soluble upon activation prior to or during use.

The water-soluble abrasive materials can be any such material as is known in the art, preferably sea salt, mineral salts, and crystalline sugars.

The shell can be made of any material that is water-insoluble during storage and can be made water soluble upon activation prior to or during use by a change of medium property and that is compatible with the application intended. The preferred shell material is a pH sensitive material that can be dissolved by a two-part system or upon dilution with large amount of water. The preferred pH sensitive shell is preferably based on Eudragit®* polymers, which are water-insoluble if the pH of the medium is below a threshold value and water-soluble if the pH of the medium is above that threshold. The most preferred Eudragit material is Eudragit L/S, which is water-soluble when the pH of the medium exceeds 6.5. The preferred aqueous based cosmetic composition of this invention is provided in two solution portions. The first portion of the delivery system contains the water-soluble abrasive cores coated with Eudragit L/S, suspended in an aqueous formula with surfactants and a low pH (pH=6.0). A second portion of the delivery system would contain the same or different surfactants at a much higher pH so that when the two portions are mixed prior to or during use, the resulting pH would exceed 6.5 and facilitate the dissolution of the Eudragit coating and expose the abrasive salt or sucrose for exfoliation purposes. Both the core abrasive material and the shell in this invention are water soluble during application. The dilution of the first portion of the delivery system by a large amount of water also contributes to the increase of pH value over the 6.0 threshold and can be used to dissolve the shell alone or in conjunction with the second portion of the delivery system.
*Trademark of Kohm Rohm Gmbh & Co., KG, Darmstadt, Germany.

### DETAILED DESCRIPTION OF THE INVENTION

The invention provides core/shell composite particles for use in exfoliating purposes and cosmetic products containing such composite particles.

The core is a water-soluble abrasive material, which can be any material possessing these properties. The preferred material is crystalline sugars, such as sucrose, dextrose and others, or mineral salts such as sodium, potassium, or magnesium chloride, bromide and sulfate, or natural sea salts, because of their high solubility ready availability and economy. The core is preferably sphere-shaped though any other suitable shape could be used. The size of the sphere-shaped core is 10-1000 microns in diameter, and preferably 10-100 microns in diameter.

The shell, or coating, can be any material that is water-insoluble during storage, but becomes water-soluble upon activation prior to or during use. Typical shell coating polymers include polymers, copolymers and cross polymers of acrylic acid, esters of acrylic acid, vinyl and substituted vinyl polymers, polyurethanes, silicon polymers, polyquaterniums, polyesters, polyamides, insoluble proteins, natural fixatives such as rosins and shellacs, waxes, fats, hydrogenated triglycerides and other hydrophobic materials that have low solubility under the condition of final formulation. Preferred polymers include octylacrylamide copolymer (Dermacryl® 79, National Starch), VP/vinyl caprolactam/DMAPA acrylates copolymer (AquaFlex® SF40, International Specialty Products), adipic acid/DMPA/hexylene glycol/neopentyl glycol/SMDI copolymer (Avalure® 430, Noveon), and Eudragit® L/S, acrylates copolymer (Rohm Gmbh & Co., KG, Darmstadt). The activation method has to be compatible with the application desired and easy to carry out. The preferred material possesses solubility in water and is pH sensitive. The preferred pH sensitive material is an Eudragit acrylic polymer and more particularly an anionic polymer based on a methacrylic acid ester. Films or coatings of such polymers are insoluble at low pH values. They are thus resistant to gastric fluid and are used widely as coating for medicines for controlled release of the active inside human digestive tract. For example, U.S. Pat. No. 5,962,024 teaches the use of Eudragit S for coating a peroral medicine that is to be released only in the lower gastrointestinal tract, but not the stomach where the environmental pH is substantially lower. Various Eudragit polymers and their mixtures are available from Rohm Pharma Polymers for dissolution in water and the subsequent release of enclosed/coated active when the environmental pH values exceed a tunable threshold from 5.5 to 7.0. The preferred Eudragit polymer for the purpose of skin exfoliating composition is the mixture of Eudragit L 100 and Eudragit S 100, abbreviated as Eudragit L/S, which gradually dissolves in water in a pH range from 6.0 to 6.5 and above.

Plasticizers and excipients may be added to the coating solution to modify the shell's resistance to solubility or hardness and dyes and/or pigments may be added for decorative purposes.

The dry shell thickness is in the range of 1-500 microns and preferably 10-100 microns.

The composite particles are manufactured by coating particulate cores with a coating solution containing the appropriate polymers using any suitable conventional coating means known in the art.

Examples of suitable formulations and coating methods are given as follows (amounts given by weight percentage except in Formula E). The coating method following each formulation is not restricted to that formulation and can be applied to other formulations whenever it is appropriate.

Formula A:

| | Amount |
|---|---|
| Material | (% wt/wt) |
| acrylates/octylacrylamide copolymer (Dermacryl ® 79, | 2 |
| National Starch) | |
| ethanol | 48 |
| sucrose sanding sugar (Indiana Sugars) | 50 |

The polymer solution is sprayed onto the fluidized sucrose at slightly elevated temperature (3040° C.).

Formula B:

| Material | Amount (% wt/wt) |
|---|---|
| Paraffin Wax (Strahl and Pitsch) | 30 |
| parts sanding sugar (Indiana Sugars) | 70 |

The paraffin and sugar are fluidized together at 20-25° C. The temperature is ramped to a few degrees above the melting point of the paraffin and then cooled back to 20° C.

Formula C:

| | Amount |
|---|---|
| Material | (% wt/wt) |
| VP/vinyl caprolactam/DMAPA acrylates copolymer | 5 |
| (AquaFlex ® SF-40, International Specialty Products) | |
| dimethicone | 1 |
| ethanol | 44 |
| sea salt (Refined Sea Salt # 1120, Morton) | 50 |

The polymer solution containing the dimethicone is sprayed onto to a fluidized bed of the sea salt at ambient temperature. Fluidization is continued until the ethanol is completely flashed off

Formula D:

| Material | Amount (%, wt/wt) |
|---|---|
| adipic acid/DMPA/hexylene glycol/neopentyl | 6 |
| glycol/SMDI copolymer (Avalure ® 430, Noveon) | |
| isopropanol | 54 |
| sea salt (Refined Sea Salt # 1120, Morton) | 40 |

The polymer solution is sprayed onto a fluidized bed of the sea salt at a slightly elevated temperature (30-45° C.).

Formula E:

| Material | Amount (grams) |
|---|---|
| Eudragit L/S | 5.00 |
| triethyl citrate | 0.05 |
| talc | 2.00 |
| pigment dispersion | 0.95 |
| ethyl alcohol | 92.0 |
| fine crystalline sucrose | 200 |

The Eudragit polymer, triethyl citrate plasticizer, talc and pigment dispersion are mixed and subsequently sprayed onto the sucrose at a slightly elevated temperature (30-40° C.).

The particles can be supplied in aqueous solutions with surfactants and the pH value is adjusted to 6.0 using an acid. The surfactant can be any conventionally used anionic, cationic, nonionic, zwitterionic or amphetoric surfactant or mixtures thereof, and is preferably a sodium C14-16 olefin sulfonate. The acid for adjusting the pH value can be any conventionally used organic or inorganic acid or mixtures thereof, and is preferably citric acid. The solution can optionally contain other excipients, for example, foam boosters such as diethyl lauramide, preservatives and antioxidants such as BHT, fragrances, dyes, and most particularly gelling agents for preventing the sedimentation of the particles and facilitating the formation of a homogeneous suspension, such as the inorganic and organic gelling agents disclosed in U.S. Pat. No. 4,704,222.

Examples of the formulation of aqueous solution containing the particles are given as follows:

### EXAMPLE 1

### Skin Polisher, Mild Exfoliator

| Material | Amount (% wt/wt) |
|---|---|
| Water | 47.77 |
| Disodium EDTA | 0.02 |
| Glycerin 99%, USP | 7.36 |
| Sodium Borate | 0.92 |
| C12-C15 alkyl benzoate | 16.75 |
| Stearic acid | 2.00 |
| Dimethicone 100 cps | 1.00 |
| Stearic acid | 3.68 |
| Ethylene glycol distearate | 1.00 |
| Cetyl Alcohol | 1.00 |
| PEG-10 (Soya Sterol) | 0.50 |
| Castor Oil (Stearyl alcohol and PEG-40) | 0.50 |
| Propyl paraben | 0.50 |
| Jojoba oil | 2.80 |
| Mineral Oil | 3.70 |
| Coated Sugar from Formula “E” | 10.50 |

### EXAMPLE 2

### Foot Scrub

| Material | Amount (%, wt/wt) |
|---|---|
| Water | 68.25 |
| Lubrajel IIXD | 12.00 |
| TEA Lauryl Sulfate (40%) | 4.00 |
| Propylene glycol | 2.50 |
| Glycerine | 2.50 |
| Peppermint (Mentha) Piperita Oil | 0.10 |
| Actiphyte of Chamomile | 0.05 |
| Actiphyte of Ivy | 0.05 |
| Polymethoxy Bicyclic Oxazolidine | 0.20 |
| Coated sea salt from formula “C” | 10.00 |
| D&C Red No. 33 | 0.35 |

### EXAMPLE 3

### Shower Gel/Body Scrub

| | Amount |
|---|---|
| Material | (%, wt/wt) |
| Water | 60.00 |
| Acrylates/Vinyl isodecanoate Crosspolymer | 1.00 |
| Imidazolidinyl urea | 0.30 |
| Methylchloroisothiazoline and methylisothiazolinone | 0.05 |
| Sodium hydroxide | 3.00 |
| Sodium laureth sulfate | 20.00 |
| Tegobetaine L5351 | 5.00 |
| PEG-40 Hydrogenated Castor oil | 0.60 |
| Perfume | 0.05 |
| Coated Sugar from Formula “A” | 10.00 |

The aqueous solution containing the surfactants and particles can be used alone for the purpose of skin exfoliation. The dilution of the solution with large amount of water during use will cause the pH of the medium to increase beyond the 6.5 dissolution threshold of the coating material and leads to a gradual dissolution of the coating material. The mechanical shearing force in the rubbing motion during exfoliation will also facilitate the breakage of the coating material. The two factors combined will cause the gradual exposure of the abrasive core for efficient skin exfoliation. Upon contact with water, the core also starts dissolution, and both the core and the coating will be completely dissolved either at the end of the use or shortly after, leaving no solid residue in the bath tub, sink basin or shower floor.

A more preferred method for activating the aqueous solution containing the particles is by supplying a second portion of aqueous solution which possesses a pH value higher than the first portion. For example, the second portion can be an aqueous solutions of the same or different surfactants adjusted and buffered to a pH of 7.5. When the two portions are mixed in equal proportions, the resulting pH of the mixture will exceed the dissolution threshold of the Eudragit L/S in the coating. Alternatively the second portion can be supplied in fewer amounts but adjusted to a pH high enough that upon mixing with the larger amounts of first portion, the resulting pH of the mixture exceeds the dissolution threshold of the Eudragit L/S in the coating. The mixture is applied to the skin for exfoliation immediately after the two portions are combined and thoroughly mixed.

An example of this two-portion system is given as follows,

### EXAMPLE 4

### A pH Activated Two-Portion Exfoliant

| | |
|---|---|
| Material | Amount (%, wt/wt) |

Portion A.

| | |
|---|---|
| Water | 60.00 |
| Acrylates/Vinyl isodecanoate crosspolymer | 1.00 |
| Imidazolidinyl urea | 0.30 |
| Methylchloroisothiazoline and | 0.05 |
| methylisothiazolinone | |
| Sodium hydroxide | 3.00 |
| Sodium laureth sulfate | 20.00 |
| Tegobetaine L5351 | 5.00 |
| PEG-40 hydrogenated castor oil | 0.60 |
| Perfume | 0.05 |
| Coated sugar from Formula “A” | 20.00 |
| Citric acid to adjust pH to 5.2 | QS |

Portion B.

| | |
|---|---|
| Water | 60.00 |
| Acrylates/Vinyl isodecanoate crosspolymer | 1.00 |
| Imidazolidinyl urea | 0.30 |
| Methylchloroisothiazoline and | 0.05 |
| methylisothiazolinone | |
| Sodium hydroxide | 3.00 |
| Sodium laureth sulfate | 20.00 |
| Tegobetaine L5351 | 5.00 |
| PEG-40 hydrogenated castor oil | 0.60 |
| Perfume | 0.05 |
| Sodium hydroxide to adjust pH to 7.7 | QS |

In the preferred method, where two aqueous portions are supplied and they are combined before use, the two portions can be supplied in separate containers or in separate compartments in the same container. When the latter dual compartment containers are employed, a means for mixing before use is provided. Such dual compartment containers are well known in the art, such as those disclosed in U.S. Pat. Nos. 2,859,898; 3,305,368; 3,347,410; 3,425,598; 3,458,076; 3,891,125; 3,924,741; 4,903,865; and 5,246,142.

It is within the spirit of this invention to contemplate other formulations containing the above-identified pH sensitive core/shell particles for exfoliating purposes, such as in the form of cosmetic bars, creams and etc.

I claim:

1. Composite particles possessing a core/shell structure, wherein the core comprises a water-soluble abrasive material, and the shell comprises a material whose solubility in water substantially depends on the properties of the medium wherein the composite particles are present.

2. The composite particles according to claim 1, wherein said water soluble abrasive material is selected from the group consisting of sugars, mineral salts and sea salts.

3. The composite particles according to claim 2, wherein said water soluble abrasive mineral salt material is selected from the group consisting of sodium, potassium, and magnesium chloride, bromide and sulfate.

4. The composite particles according to claim 1, wherein said water soluble abrasive material is substantially spherical with a diameter of 10 to 1000 microns.

5. The composite particles according to claim 1, wherein said shell material possesses a solubility in water dependent on the pH of the said medium.

6. The composite particles according to claim 1, wherein said shell possesses a thickness of 1 to 500 microns.

7. The composite particles according to claim 5, wherein said shell material is water-insoluble when the pH of said medium is below a threshold, and is water-insoluble when the pH of said medium is above the said threshold.

8. The composite particles according to claim 7, wherein said shell material comprises Eudragit L/S and said pH threshold is about 6.5.

9. A cosmetic composition for exfoliating skin, comprising
a) a surfactant,
b) composite particles according to claim 1,
c) water,
d) an acid in an amount for adjusting the pH to about 6.0,
e) at least one excipient.

10. A cosmetic composition for exfoliating skin, comprising
A. a first liquid portion comprising
a) a surfactant,
b) composite particles according to claim 1,
c) water
d) an acid in an amount for adjusting the pH to about 6.0, excipients, and
B. a second liquid portion comprising
a) a surfactant,
b) water,
c) an alkaline agent in amount for adjusting the pH to about 7.

11. A method of exfoliating skin, comprising the steps of
a) applying the cosmetic composition according to claim 9 to the skin,
b) rubbing the particles in contact with the skin and,
c) washing the exfoliated skin with large amount of water.

12. a method of exfoliating skin, comprising the steps of
a) combining prior to use said first and second liquid portions according to claim 10 to form a mixture,
b) applying the mixture to the skin,
c) rubbing the particles in contact with the skin and,
c) washing the exfoliated skin with a large amount of water.
